(19) United States
(12) Patent Application Publication
(10) Pub. No.: US 2004/0052734 A1
(43) Pub. Date: Mar. 18, 2004

(54) Process for providing a stable crystalline form of salbutamol

(75) Inventors: Katharina Brodka-Pfeiffer, Mainz (DE); Peter Grass, Ingelheim (DE); Heribert Haeusler, Klein-Winternheim (DE); Peter Langguth, Biebergemuend (DE); Herbert Thieme, Ingelheim (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/617,546

(22) Filed: Jul. 10, 2003

Related U.S. Application Data
(60) Provisional application No. 60/408,375, filed on Sep. 5, 2002.

(30) Foreign Application Priority Data
Jul. 12, 2002 (EP) 02015 701.2

(51) Int. Cl. C13K 1/10; A61K 9/14; A61L 9/04

(52) U.S. Cl. 424/46; 23/295 R; 564/349

(57) ABSTRACT

The invention relates to a process for providing a stable crystalline form of a fine-milled salbutamol sulfate, which can be produced, stored and used while maintaining the aerodynamic properties required for inhalation thereof, which comprises the steps of

a) micronizing salbutamol sulfate into a particle size required for inhalation;

b) conditioning said salbutamol sulfate by treatment with a water-containing vapor; and

c) drying the substance.

### RELATED APPLICATIONS

Benefit of U.S. Provisional Application Serial No. 60/408,375, filed on Sep. 5, 2002 is hereby claimed.

### FIELD OF THE INVENTION

The invention relates to a process for providing a stable crystalline form of a fine-milled salbutamol sulfate, which can be produced, stored and used while maintaining the aerodynamic properties required for inhalation thereof, which comprises the steps of

a) micronizing salbutamol sulfate into a particle size required for inhalation;

b) conditioning said salbutamol sulfate by treatment with a water-containing vapor; and

c) drying the substance.

### BACKGROUND OF THE INVENTION

Micronisation is a high energy process, which induces changes in the crystallinity of materials. Preferably salbutamol sulphate is micronised by an air jet mill. As a result, the crystalline structures on the particles' surface are being destroyed and amorphous areas are formed. After micronisation of salbutamol sulphate only small amounts of amorphous material are produced. Nevertheless even these small amounts can have important effects on the physical stability of the powder. The amorphous state is thermodynamically unstable and tends to convert to the stable, crystalline state. The recrystallisation process of disordered regions on the particles' surface leads to agglomeration of milled particles. It is possible, that in this case bridges are being built between the individual particles, which leads to particle growth. That is an undesirable process, since particles are designed to range between 1 to 10 μm in diameter in order to exert respirative effect.

In some pharmaceutical processes such as grinding, wet granulation, tablet compaction and spray drying, disorder of crystal structure can occur and lead to amorphous particles or parts thereof. These amorphous regions can be desirable, since e.g. enhanced dissolution and a higher bioavailability may result. On the other hand, they may be disadvantageous because they may lead to a decrease of physical stability.

In various publications the importance of amorphous parts in crystalline powders has been discussed [4, 8, 9]. Amorphous regions have the rheological property of a solid state but the structure of a liquid [7]. They are thermodynamically unstable and tend to convert to a stable, crystalline state. Amorphous solids are in general physically and chemically less stable than the corresponding crystals because they are in a higher energy state than the crystalline form and they have greater molecular mobility.

Amorphous salbutamol sulphate may be formed as a result of micronisation with an air-jet-mill. Depending on the level of grinding energy, increasing portions of amorphous content may be generated on the surface of the crystal. The powder's surface is essential because the interaction between the amorphous region and other phases are different than the interaction between the phases and the material in the crystalline state.

For the crystallization process, the temperature of glass transition (Tg) is important. Above this point the molecular mobility is higher and therefore the re-crystallization process is accelerated. Tg is dependent on factors such as temperature and humidity. A greater difference between the Tg and the surrounding temperature of the sample stabilizes the amorphous state [5]. Therefore, lowering the Tg by means of a plastisizer (in most cases absorption of water) or increasing the surrounding temperature decreases the energy barrier for re-crystallization.

When the re-crystallization process occurs in an uncontrolled manner it may lead to significant problems during storage of pharmaceutical powders, i.e. uncontrolled particle growth. Thus the effectiveness of powders for inhalation may be compromised.

Therefore the problem underlying the present invention was to provide a method which allows to produce stable micronised salbutamol sulphate in which the tendency of uncontrolled particle growth has been minimized.

It has been found surprisingly, that stable micronised salbutamol sulphate can be obtained, if the micronised product is conditioned under well defined temperatures and relative humilities.

### SUMMARY OF THE INVENTION

Accordingly the present invention relates to a process for providing a stable crystalline form of a fine-milled salbutamol sulfate, which can be produced, stored and used while maintaining the aerodynamic properties required for inhalation thereof, which comprises the steps of

a) micronizing by any conventional method salbutamol sulfate into a particle size 4.6 μm;

b) conditioning said salbutamol sulfate by treatment with a water-containing vapor phase at a temperature from 20 to 50, in particular 25 to 40° C., and a relative humidity from 45 to 80%, in particular 45 to 75%, more preferably 55 to 75%; and

c) drying the substance.

### BRIEF DESCRIPTION OF THE DRAWINGS

FIG. 1 shows the isothermal microcalorimetry of a freshly micronised powder (1) and one of a conditioned powder at a temperature of 70° C. for 5 hours (2).

FIG. 2 provides the X-ray powder diffractometry of 100% amorphous material at different temperatures under vacuum.

FIG. 3 shows the particle size distributions of micronised, dry conditioned (left), wet conditioned (right) and stored powder for 4 weeks.

FIG. 4 shows the relative humidity in a PE-bag filled with a freshly micronised salbutamol sulphate powder and stored at relative humidity of 45%.

FIG. 5. provides the amorphous amounts of the stored (four weeks) sample: thermal conditioned powders in a PE-bag 1.2% (amorphous), freshly micronised powder in a PE-bag (dash) and in PE-bag+aluminium bag (dot).

FIG. 6. shows the microcalorimetry of micronised salbutamol sulphate upon storage in an opened TWO-Glass at room temperature (21.5° C., 42% RH): amorphous amounts after 1^{st} week 6.5% (3), 2^{nd} week 5.4% (2) and 4^{th} week 4.5% (1).

FIG. 7. Shows the relative humidity of a micronised and wrapped up sample of salbutamol sulphate stored at relative humidity of 55%.

### DETAILED DESCRIPTION OF THE INVENTION

The present invention relates to a process for providing a stable crystalline form of a fine-milled salbutamol sulfate, which can be produced, stored and used while maintaining the aerodynamic properties required for inhalation thereof, which comprises the steps of

a) micronizing by any conventional method salbutamol sulfate into a particle size required for inhalation, more than 90% of the particle size distribution being less than 4.6 μm;

b) conditioning said salbutamol sulfate by treatment with a water-containing vapor phase at a temperature from 20 to 50, in particular 25 to 40° C., and a relative humidity from 45 to 80%, in particular 45 to 75%, more preferably 55 to 75%; and

c) drying the substance.

In a preferred embodiment of the present invention the process may be performed in a one-step procedure or a multi-step procedure using different relative humidity/temperature combinations.

Further preferred embodiments are processes wherein:

(A) step b) is carried out at a temperature from 25 to 40° C. and at a relative humidity from 55 to 75%; and/or

(B) the said salbutamol sulfate obtained in step a) has the following particle size distribution:

| Amount [%] | Particle size [μm] |
|---|---|
| 10 | <0.70 |
| 50 | <1.53 |
| 90 | <3.42 |

and/or

(C) the said salbutamol sulfate obtained in step b) has the following particle size distribution:

| Amount [%] | Particle size [μm] |
|---|---|
| 10 | <0.75 to <0.85 |
| 50 | <1.66 to <1.80 |
| 90 | <3.55 to <3.75 |

and/or

(D) the conditioning step b) is carried out for at least 1 hour, preferably for 3 to 48 hours, more preferably for 5 to 24 hours.

Furthermore, the invention relates to a salbutamol sulfate produced by the process according to the present invention.

Another aspect of the present invention is the use of salbutamol sulfate produced by the process according to the present invention for the preparation of a medicamentation for the treatment of respiratory diseases, in particular asthma and/or COPD.

Following conditioning the powders are stored and evaluated at specific time intervals. For comparison, a sample of the micronised powder is stored without a preceding conditioning procedure and evaluated accordingly.

1. Materials and Methods

1.1. Materials

Salbutamol sulphate or albuterol sulphate is α-[[(1,1-dimethylethyl)-amino]methyl-4-hydroxy-1,3-benzenedimethanol belongs to the class of β-2-agonists and is of high commercial interest. The chemical structure is shown in formula I:

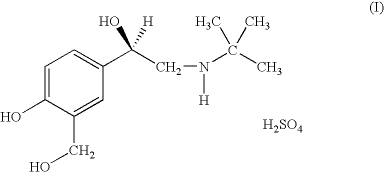

As packaging materials, a polyethylen bag or a polyethylen bag in an aluminium bag or a twist-off-Glass were used.

1.2. Methods

1.2.1. Milling

Micronised powders were prepared with a MC Jetmill 50 (Jetpharma, Balerna, Switzerland).

Extant room conditions were 21° C.±1° C. and 45%±2% RH.

1.2.2. Conditioning

The micronised powder underwent different conditioning settings in a climate chamber (Weiβ Klimatechnik GmbH, Reiskirchen-Lindstruth, Germany). The temperature was set at 25° C. and 40° C. and the relative humidity was varied in a field between 45% and 75%, respectively.

For “dry” conditioning settings the powders were prepared at a temperature of 70° C. in a thermal oven (Heraeus, Hanau, Germany).

1.2.3. Recording of Humidity

Using a humidity sensor (Ebro, Ingolstadt, Germany) the sorption behaviour of the micronised and wrapped powder was measured.

1.2.4. Particle Size Analysis

The particle size distributions of salbutamol sulphate were measured using powder laser diffraction, HELIOS-SYSTEM (Sympatec, Clausthal-Zellerfeld, Germany). Samples were introduced through the RODOS dry powder feeder. The supply pressure of the injector was at 3 bar. The optical concentration reached values between 4 and 8%.

1.2.5. Particle Morphology

The morphology of salbutamol sulphate was examined by using a DSM 926 scanning electron microscope (Zeiss, Jena, Germany). The powders were mounted onto a plate and were sputter coated with 60 nm gold/palladium.

1.2.6. Isothermal Microcalorimetry

The powder was investigated using a Thermal Activity Monitor (Type 2277, Thermometric, Sweden) at 25° C. The samples were weighted into a glass ampoule and a tube was added containing a saturated salt solution. The ampoule was sealed and equilibrated in the calorimeter for 5 min before lowering it into the measuring site.

1.2.7. Powder X-ray Diffraction

The Powder X-Ray Diffraction (Bruker, Rheinstetten, Germany) patterns were acquired at different temperatures using Cu-K α radiation (λ=1.5406 Å). The data were collected over an angular range of 2-40°2θ using a step size of 0,014°2θ and a step time of 2 sec.

2. Results and Discussion

2.1. Conditioning

2.1.1. Conditioning with Elevated Temperature (Dry Conditioning)

Conditioning of the micronised salbutamol sulphate at 70° C. for 5 hours does not lead to recrystallisation, and therefore no stable product. Rather, such “conditioning” conditions are counterproductive, since due to the high temperature water is expelled from the sample. Water, however, serves as a plasisizer and consequently its disappearance leads to a stabilisation of the amorphous state (increase in Tg). Therefore, no changes in the amorphous content are observed under such conditions.

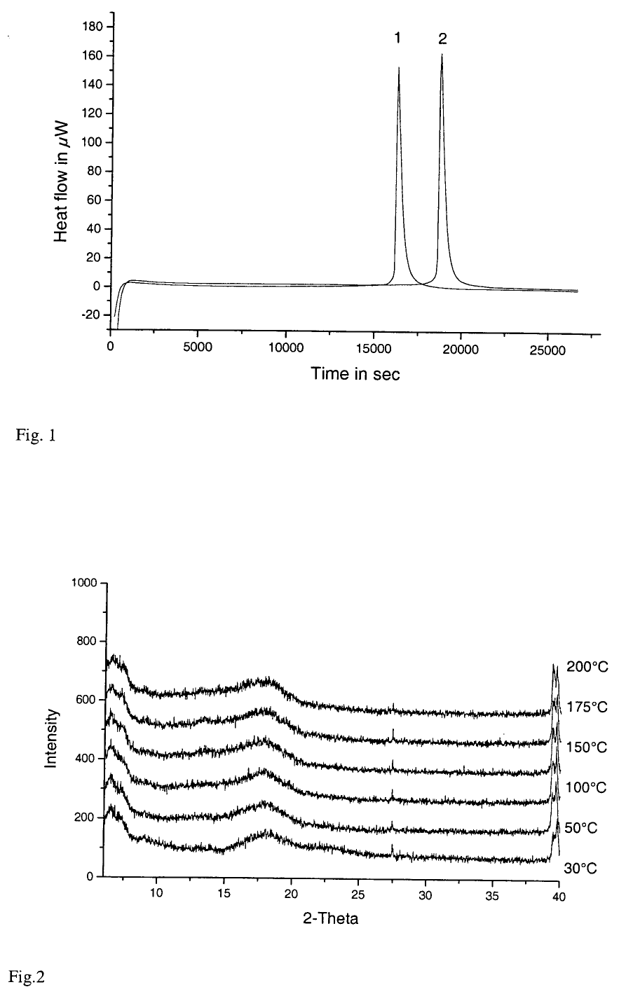

Isothermal microcalorimetry shows that the exothermal recrystallisation process is delayed on account of the water displacement (FIG. 1). An increase of the thermal conditioning time up to 24 hours has no additional effect.

Particle growth is not observed following dry conditioning. This is shown in the following Table 1.

TABLE 1
Particle size distribution after thermal conditioning of micronised
salbutamol sulphate

| | | Micronised powder | relative change of |
|---|---|---|---|
| | | conditioned 5 hours | particle size |
| | Micronised powder | by 70° C. | spreading |
| | μm | μm | % |
| 10% < | 0.83 | 0.82 | −1.2 |
| 50% < | 1.94 | 1.90 | −2.1 |
| 90% < | 4.53 | 4.45 | −1.8 |

Using x-ray powder diffractometry in vacuum and under different temperatures (FIG. 2), it can be proved, that amorphous salbutamol sulphate (produced by freeze-drying) does not recrystallize under exclusion of humidity. Water molecules are therefore necessary for the transformation into the thermostable state. Consequently, minimum humidities are essential in order to put the recrystallisation process into operation.

3.1.2 Conditioning with Moisture (Wet Conditioning).

Relative humidities (RH)<50% at 25° C. are insufficient to achieve complete recrystallisation of amorphous salbutamol sulphate within 24 hours. For example, conditioning of a sample at 25° C. and 45% RH for 24 hours leads to a reduction of the amorphous fraction by 2.5% (initial sample amorphous content of 7.7%). This can be shown by the isothermal microcalorimetry.

When sufficient conditioning settings are employed, particle growth occurs in each batch. This particle growth cannot be excluded since a formation of bridges between the amorphous surfaces takes place during the recrystallisation process.

Through selective variation of the humidity and temperature, the percentage of particle growth in dependence on these factors should be evaluated. A clear tendency towards stronger particle growth can be observed by an increase of humidity. An increase of the temperature up to 40° C. with constant relative humidities also shows a tendency towards particle growth, however, this dependence is not very expressed (Tab. 2).

TABLE 2
Dependence of the particle growth on the humidity and temperature
settings during conditioning

| | | |
|---|---|---|
| | | relative change in |
| | Micronised powder | particle size |
| Micronised powder | conditioned | distribution |
| μm | μm | % |

24 h 55% 25° C.

| | | | |
|---|---|---|---|
| 10% < | 0.70 | 0.78 | 11.4 |
| 50% < | 1.30 | 1.66 | 8.50 |
| 90% < | 3.42 | 3.56 | 4.10 |

24 h 65% 25° C.

| | | | |
|---|---|---|---|
| 10% < | 0.70 | 0.82 | 17.1 |
| 50% < | 1.53 | 1.76 | 15.0 |
| 90% < | 3.42 | 3.67 | 7.30 |

24 h 75% 25° C.

| | | | |
|---|---|---|---|
| 10% < | 0.70 | 0.83 | 18.6 |
| 50% < | 1.53 | 1.77 | 15.7 |
| 90% < | 3.42 | 3.69 | 7.90 |

24 h 55% 40° C.

| | | | |
|---|---|---|---|
| 10% < | 0.70 | 0.79 | 12.9 |
| 50% < | 1.53 | 1.68 | 9.80 |
| 90% < | 3.42 | 3.59 | 5.00 |

24 h 65% 40° C.

| | | | |
|---|---|---|---|
| 10% < | 0.70 | 0.80 | 14.3 |
| 50% < | 1.53 | 1.72 | 12.4 |
| 90% < | 3.42 | 3.64 | 6.40 |

24 h 75% 40° C.

| | | | |
|---|---|---|---|
| 10% < | 0.70 | 0.84 | 20.0 |
| 50% < | 1.53 | 1.78 | 16.3 |
| 90% < | 3.42 | 3.69 | 7.90 |

Following five hours of conditioning at 40° C. and 75% RH turns a sample of the partially amorphous powder into complete crystalline material. Additional duration of conditioning beyond five hours shows no effect on the particle size, as does storage under the same conditions for two weeks. These observations reveal that the aim of the conditioning process is already reached after five hours. Further conditioning time has no influence on the physical stability of the powder (results not showed).

In summary it has been shown that pure thermal conditioning is not practical and relative humidity at room temperature should be at least 55%, so that the product is able to recrystallize within 24 hours. This humidity level shows the smallest influence on particle growth. If the duration of conditioning is an issue, e.g. 40° C. and 75% RH are suitable to obtain a stable, entirely crystalline product already after five hours.

3.2. Storage

3.2.1. Storage Following Conditioning

The stability of the product is dependent on the methods of conditioning.

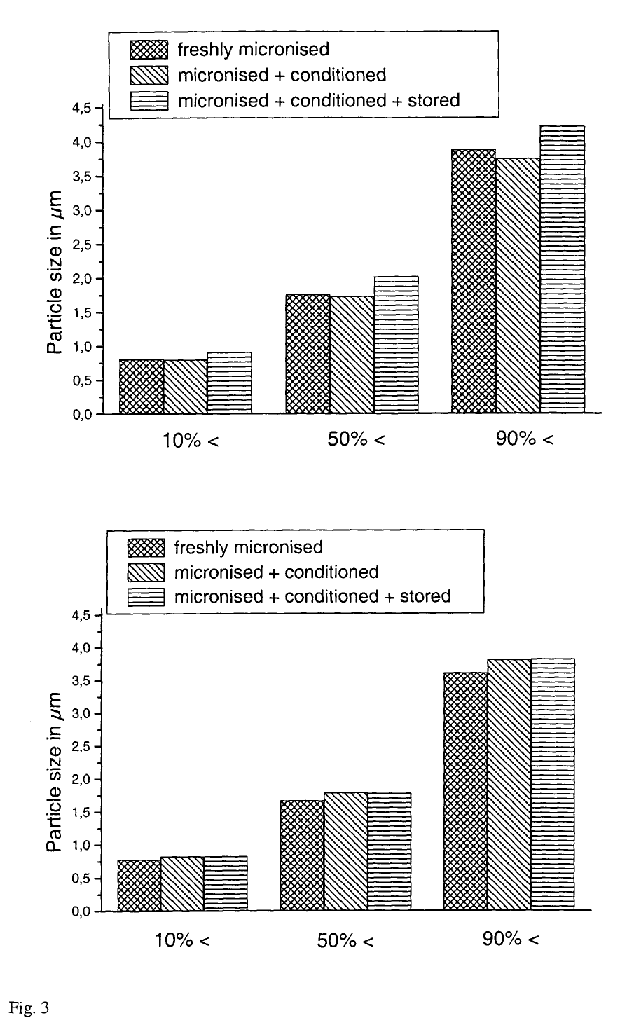

With dry conditioning, uncontrolled particle growth up to 16% in terms of particle diameters was observed following four weeks of storage. Wet conditioning, however, produces products which remain stable throughout the period of storage (FIG. 3).

3.2.2. Storage without a Conditioning Step

In order to assess particle stability without a previous conditioning step, freshly micronised material is wrapped and stored between 21° C. and 23° C. and a relative humidity of approx. 45% for a total period of three months without a preceding conditioning step.

After one, two and four weeks as well as after three months a sample is collected and analysed for its particle size distribution and amorphous content.

It can be shown that the wrapped and stored powder without preceding conditioning, shows likewise particle growth (results not showed).

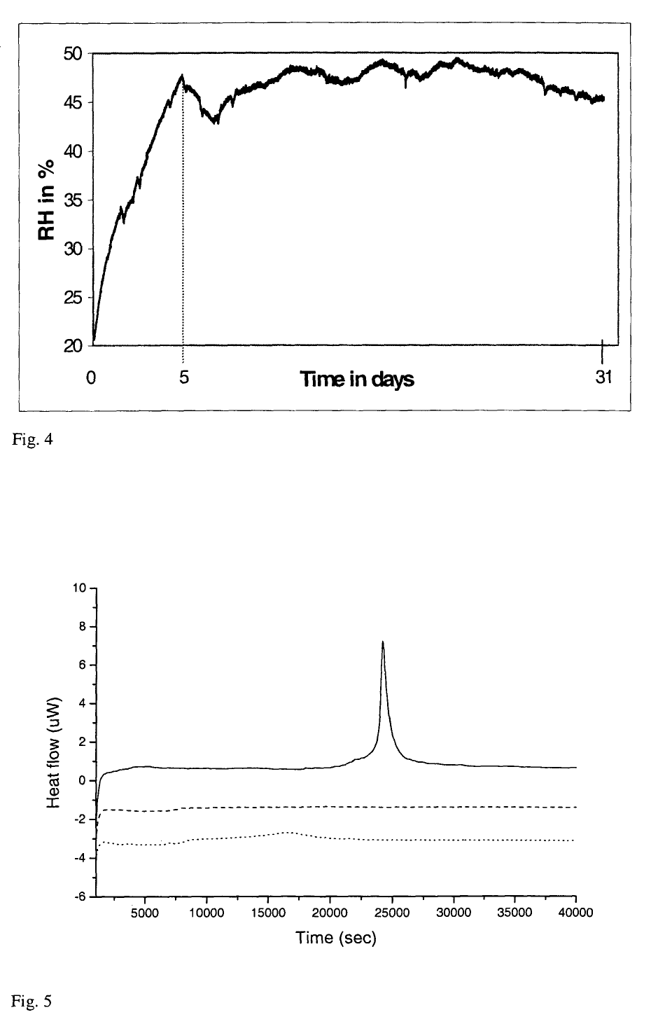

With the help of a humidity sensor it is possible to observe the sorption behaviour of the micronised, in a PE-bag wrapped, powder. Approx. 30 g of the freshly grinded substance are wrapped in a PE-bag together with a humidity sensor and afterwards closed. Analysis of the water-sorption behaviour of micronised salbutamol sulphate stored within a PE-bag is shown in FIG. 4.

At first there is a low relative humidity in the closed bag, since the water molecules are being adsorbed by the amorphous areas on the surface of the particles. Within five days ambient humidity is reached inside the bag. Since the relative humidity is less then 50%, the process of recrystallisation takes place only at a comparatively slow rate. The time to equilibrium is approximately five days. The recrystallisation does not take place as a cooperative process. Rather, the amorphous material assimilates water, recrystallizes and the desorbed water molecules are being assimilated by further amorphous regions. This can be interpreted from the oszillating shape of the plot in FIG. 4.

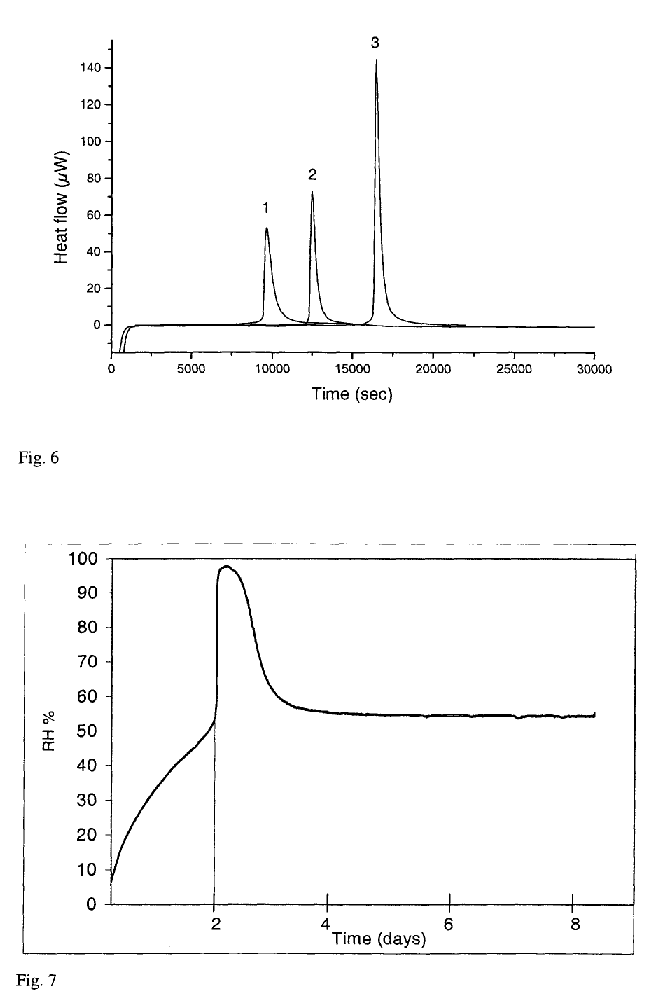

The stored material is tested for amorphous amounts, too. A continuous decrease of the amorphous content becomes obvious, whereas the powder which is wrapped in a PE-bag plus an aluminium bag shows the highest amorphous content after a period of two weeks. After four weeks the amorphous content of the unconditioned powder lies beneath 0.5%. The amorphous amount of the thermal conditioned powder has still after four weeks of storage a value of circa 1.2% (FIG. 5). FIG. 6 shows the continous decrease of amorphous amounts of micronised powder which is stored in an open TWO-Glass at room conditions. In this case the process of recrystallisation is no cooperative, too.

An analogous storage experiment is carried out at the same temperature level but at a higher humidity of 55% (FIG. 7). Here, a clear difference in the sorption behaviour becomes evident. The relative humidity increases up to approx. 55%, followed by a quick recrystallisation (cooperative), desorption and adjustment of the equilibrium-moisture content to approx. 55%. Under such conditions, the sample is already completely recrystallized within one week. The relative humidity reaches in a wrapped state occasionally values of more than 90%.

In summary it can be shown that during storage of the micronised powder a “self-conditioning” process may occur. The rate of self-conditioning is dependent on the relative humidity. Lower humidity leads to an extended duration of self-conditioning process. Furthermore, when particles are stored below a relative humidity of 50%, a significantly lower particle growth is observed as compared to particles which are stored above this humidity value.

3. Conclusion

Sufficient conditioning of salbutamol sulphate ensures complete conversion of amorphous parts into crystalline material and stabilisation of the powder. The physical stability of the micronised powder is influenced by the conditioning parameters.

Dry conditioning has been shown to be useless in this regard. The relative humidity at room temperature should be at least 55%, so that the powder will recrystallize within a period of 24 hours. This humidity level shows the smallest particle growth under the conditions tested. When shorter conditioning periods are desired, e.g. 40° C. and 75% R.H. are alternative conditions to obtain a stable, entirely crystalline product.

The advantage of optimized conditioning process parameters is that every batch can be controlled to show a relatively small acceptable particle growth. Particle growth cannot be completely prevented, however, since bridges are being built between the amorphous surfaces during the process of re-crystallization.

The extent of the agglomeration is dependent on the amorphous parts and therefore on the micronisation energy, but also on the parameters of conditioning and storage. The relative humidity has a strong impact on the particle growth. Raised temperature in the range of 25° C. to 40° C. has a less pronounced effect, whereas conditioning time is insignificant.

Non conditioned micronised powder undergoes a _{“}self-conditioning” process upon storage. The kinetics of the process is depending on the relative humidity with higher humidities favouring faster rates.

A steady particle size growth is seen with unconditioned product. The powder which is stored below 50% R.H. shows a significantly smaller growth, than one which is stored above this humidity level.

Influences of conditioning and storage may be assessed with isothermal micro-calorimetry and the DVS-method, according to the determination of freshly micronised material.

References

1. Ahlneck, C., Zografi, G., 1990, The molecular basis of moisture effects on the physical and chemical stability of drugs in the solid state. Int.J.Pharm. 62, 87-95

2. Briggner, L.-E., Buckton, G., Bystrom, K., Darcy, P., 1994. The use of isothermal microcalorimetry in the study of changes in crystallinity induced during processing of powders. Int. J. Pharm. 105, 125-135.

3. Elamin,A. A., Sebhatu,T., Ahlneck, C., 1995. The use of amorphous substances to study mechanically activated materials in the solid state. Int.J.Pharm. 119, 25-36

4. Hancock,B., Zografi,G., 1997. Characteristics and significance of the amorphous state in pharmaceutical systems. J. Pharm. Sci. 86, 1-12

5. Saleki-Gerhardt,A., Ahlneck, C., Zografi, G., 1994. Assessment of disorder in crystalline solids. Int. J. Pharm. 101, 237-247

6. Sebhatu, T., Angberg, M., Ahlneck, C.,1994. Assesment of the degree of disorder in crystalline solids by isothermal microcalorimetry. Int. J. Pharm. 104, 135-144

7. Threlfall, T. L., 1995. Analysis of organic polymorphs. Analyst, Vol. 120

8. Ticehurst M. D., Rowe R. C., York P., 1994. Determination of the surface properties of two batches of salbutamol sulfate by inverse gas chromatography. Int.J.Pharm., 111, 241-249

9. Ward, G. H., Schultz K., 1995. Process-induced crystallinity changes in albuterol sulfate and its effect on powder physical stability. Pharm. Research, 12(5), 773-779

What is claimed is:

1. A process for providing a stable crystalline form of a fine-milled salbutamol sulfate, which can be produced, stored and used while maintaining the aerodynamic properties required for inhalation thereof, which comprises the steps of
a) micronizing by any conventional method salbutamol sulfate into a particle size required for inhalation, more than 90% of the particle size distribution being less than 4.6 μm;
b) conditioning said salbutamol sulfate by treatment with a water-containing vapor phase at a temperature from 20 to 50° C. and a relative humidity from 45 to 80%; and
c) drying the substance.

2. The process according to claim 1 wherein the conditioning, in the case of a substance mixture, may be performed in a one-step procedure or a multi-step procedure using different relative humidity/temperature combinations.

3. The process according to claim 1 wherein step b) is carried out at a temperature from 25 to 40° C. and at a relative humidity from 45 to 75%.

4. The process according to claim 1 wherein salbutamol sulfate obtained in step a) has the following particle size distribution:

| Amount [%] | Particle size [μm] |
|---|---|
| 10 | <0.70 |
| 50 | <1.53 |
| 90 | <3.42 |

5. The process according to claim 1 wherein salbutamol sulfate obtained in step b) has the following particle size distribution:

| Amount [%] | Particle size [μm] |
|---|---|
| 10 | <0.75 to <0.85 |
| 50 | <1.66 to <1.80 |
| 90 | <3.55 to <3.75 |

6. The process according to claim 1 wherein the conditioning step b) is carried out for at least 1 hour.

7. Crystalline salbutamol sulfate produced by the process of claim 1.
